(19) United States
(12) Patent Application Publication
(10) Pub. No.: US 2024/0053329 A1
(43) Pub. Date: Feb. 15, 2024

(54) MULTIMODAL TESTING SYSTEM

(71) Applicant: Fluxergy Inc., Irvine, CA (US)

(72) Inventors: Farzad Izadi Kharazi, Rancho Santa Margarita, CA (US); Ryan Alan Revilla, Irvine, CA (US); Steve Hoe Lee, Irvine, CA (US); Roy James Heltsley, Irvine, CA (US); Eric Charrault, Madison, AL (US); Tej Rushikesh Patel, Carlsbad, CA (US); Jonathan Wen-Jiu Tu, Irvine, CA (US)

(21) Appl. No.: 18/178,142

(22) Filed: Mar. 3, 2023

Related U.S. Application Data
(60) Provisional application No. 63/398,063, filed on Aug. 15, 2022.

(51) Int. Cl. G01N 33/53; G01N 35/00

(52) U.S. Cl. CPC G01N 33/53; G01N 35/0092; G01N 2035/0094

(57) ABSTRACT

A multimode, multiplex assay analysis system performs multiple assays in parallel or substantially in parallel. The assays that are performed may be the same assay, similar assays or different assays. Each assay may require the use of various hardware resources. As such, the multimode, multiplex assay analysis system receives, generates, and/or updates a schedule for each hardware resource based, at least in part, on the hardware requirements and/or timing requirements associated with each assay.

### CROSS-REFERENCE TO RELATED APPLICATIONS

The present application claims priority to U.S. Provisional Application 63/398,063 entitled “Multimodal Testing System”, filed on Aug. 15, 2022, the entire disclosure of which is hereby incorporated by reference in its entirety.

### FIELD OF THE DISCLOSURE

The present application describes a multimodal multiplex assay analysis system that can perform or execute a number of similar assays and/or a number of different assays simultaneously, substantially simultaneously and/or in series.

### BACKGROUND

Conventional assay analysis systems utilized in a biochemical and/or biomedical environment typically perform a single assay at a time. For example, the conventional assay analysis system may receive a test card on which a sample has been provided. The conventional assay analysis system utilizes various hardware components to analyze the sample based on the type of assay being performed. Once the assay is complete, the assay analysis system may perform a similar assay or a different assay on another test card onto which another sample has been provided.

However, some assays that are performed by the conventional assay analysis system may take anywhere from a number of minutes to complete to a number of hours to complete. Thus, if multiple assays are to be performed, the first assay must be entirely completed before another assay can be performed. Additionally, some assays may require some hardware components while other assays may require similar hardware components or additional hardware components. As a result, one assay analysis system may be used for a first assay or set of assays while a second assay analysis system may be used for a second assay or set of assays.

Accordingly, it would be beneficial for an assay analysis system to address the drawbacks outlined above by having the capabilities to perform multiple different (or the same) assays at the same time or substantially the same time.

### SUMMARY

The present application describes a multimode, multiplex assay analysis system that performs data collection and analysis for two or more assays in parallel. For example, the multimode assay system described herein receives or is otherwise associated with a multimode consumable. In an example, the multimode consumable may be a test card or other testing apparatus on which a single sample and/or multiple samples may be placed. The multimode consumable includes instructions and/or an identification of two or more assays that are to be performed by the multimode, multiplex assay analysis system. In an example, each assay associated with the multimode consumable requires various hardware resources (e.g., image sensor, pump, temperature element, light source) of the multimode assay system at various times. As such, the multimode assay system described herein dynamically schedules the use of the various hardware resources based on the requirements of each assay. In an example, the hardware resources are scheduled such that each assay is performed in parallel, substantially in parallel and/or in series in the shortest amount of time possible.

Accordingly, the present application describes a method that includes receiving identification information associated with at least one multimode consumable and determining, based at least in part, on the identification information, a plurality of assays that are to be performed by a multimode assay system. A determination is made regarding the resource requirements for each assay of the plurality of assays. Based on the determined resource requirements, an initial resource schedule is generated. In an example, the initial resource schedule enables each assay of the plurality of assays to be performed simultaneously or substantially simultaneously.

The present application also describes a method that includes receiving at least one resource item request associated with a first assay of a plurality of assays. In an example, the plurality of assays are performed in parallel or substantially in parallel by a single multimode, multiplex assay system. When the at least one resource item request is received, a determination of an availability of the resource item is made. The determination is based, at least in part, on at least one resource schedule associated with the single multimode, multiplex assay system. The at least one resource schedule is updated based, at least in part, on the determined availability of the resource item.

Also described is a system that includes at least one processor and at least one memory communicatively coupled to the at least one processor. The at least one memory stores instructions that, when executed by the at least one processor, perform operations. In an example, the operations include receiving a first resource item request associated with a first assay. The first resource item request identifies at least one resource item. The operations also include receiving a second resource item request associated with a second assay. In an example, the second resource item request identifies the at least one resource item. Based at least in part on first timing information associated with the first resource item request and on second timing information associated with the second resource item request a determination is made regarding an availability of the at least one resource item.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following Figures.

FIG. 1A illustrates a multimode assay system that receives consumable information in order to perform multiple assays in parallel according to an example.

FIG. 1B illustrates the multimode assay system of FIG. 1A in which results of the multiple assays are provided to a computing device according to an example.

FIG. 2 illustrates a method for generating an initial resource schedule for a multimode assay system according to an example.

FIG. 3 illustrates a method for updating a resource schedule associated with a multimode assay system according to an example.

FIG. 4 is a block diagram illustrating example physical components of a computing device with which aspects of the disclosure may be practiced.

FIG. 5 is an illustrative depiction of a generalized structure of a test card in accordance with the present disclosure.

FIG. 6 is an illustrative depiction of one embodiment of a test card configured for polymerase chain reaction (PCR) testing and lateral flow assay (LFA) testing in accordance with the present disclosure.

FIG. 7 is an illustrative depiction of one embodiment of a test card configured for PCR testing and LFA testing in accordance with the present disclosure.

FIG. 8 is an illustrative depiction of one embodiment of a test card configured for LFA testing and PCR testing in accordance with the present disclosure.

FIG. 9 is an illustrative depiction of one embodiment of a test card configured for LFA testing and PCR testing in accordance with the present disclosure.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Examples may be practiced as methods, systems or devices. Accordingly, examples may take the form of a hardware implementation, an entirely software implementation, or an implementation combining software and hardware aspects. The following detailed description is therefore not to be taken in a limiting sense, and the scope of the present disclosure is defined by the appended claims and their equivalents.

The present application describes a multimode, multiplex assay analysis system (also referred to herein as a “multimode assay system” or a “multimode assay analysis system”). The multimode assay system performs various steps or operations for two or more assays in parallel or substantially in parallel. In an example, each of the two or more assays may be the same assay, similar assays, or different assays. As used herein, the same assay means that the same test is performed looking for the same target (e.g., two PCR tests each looking for COVID-19). Likewise, a “similar assay” means the same modality test, but looking for different targets (e.g., a first PCR test for COVID-19 and a second PCR test for something else). Additionally, as used herein, a “different assay” means different modalities of a test. Thus, the test may be looking for the same target or different targets (e.g., a PCR test for COVID-19 in a water sample and an electrochemical test for lead in the water sample).

Further each of the two or more assays may utilize the same hardware resources, similar hardware resources and/or different hardware resources. As will be described in greater detail herein, the multimode assay system receives information associated with or otherwise provided by at least one multimode consumable. In an example, the multimode consumable may be a test card or other testing apparatus on which a single sample and/or multiple samples may be placed. The multimode consumable includes instructions and/or an identification of two or more assays that are to be performed by the multimode assay system.

Each assay on the multimode consumable requires various hardware resources of the multimode assay system at various times. For example, each assay may require use of one or more pumps and/or valves associated with the multimode assay system. In another example, each assay may require use of a temperature controlling element of the multimode assay system. In yet another example, each assay may require use of an image sensor of the multimode assay system and/or a light source of the multimode assay system. Although specific hardware resources are mentioned, the multimode assay system may include various hardware resources and/or various sensors that may be utilized during the performance of the various assays. The various hardware resources and/or various sensors of the multimode assay system are also referred to herein as a “resource item” or “resource items.”

In some examples, two or more resource items may be used in parallel, substantially in parallel and/or in series. Additionally, two or more resource items may be used in parallel, substantially in parallel and/or in series on a single sample provided on a multimode consumable and/or on multiple samples provided on a multimode consumable. For example, two different sensors may take a measurement on at least one sample in parallel, substantially in parallel and/or in series. Further, each resource item may be associated with a single schedule or multiple different schedules generated or otherwise provided by a scheduling system.

As used herein, “in parallel” means that a time period in which one resource item is in use overlaps entirely with a time period in which the other resource item is in use. Additionally, when two of the two or more resource items are used “substantially in parallel”, this means that a time period in which one resource item is in use overlaps, at least partially, with a time period in which the other resource item is in use. When two of the two or more resource items are used “in series”, a time period in which one resource item is in use arises prior to or subsequent to a time period in which the other resource item is in use.

In order to ensure that each assay is completed in parallel, substantially in parallel and/or in series and in a desired time frame (e.g., in the shortest amount of time possible), the multimode assay system described herein dynamically schedules the use of the various hardware resources based on the requirements of each assay. For example, during the performance of each assay, the multimode assay system determines the hardware resource or hardware resources that are needed for a particular operation or step in the performance of each of the assays, as well as the timing requirements for each hardware resource. The multimode assay system then ensures that the various hardware resources are available at the requested/needed time and for the requested duration. If each assay is requesting the same hardware resource at the same time or at a similar time, the multimode assay system determines how to resolve the hardware resource conflict based on time parameters and/or a priority associated with each assay.

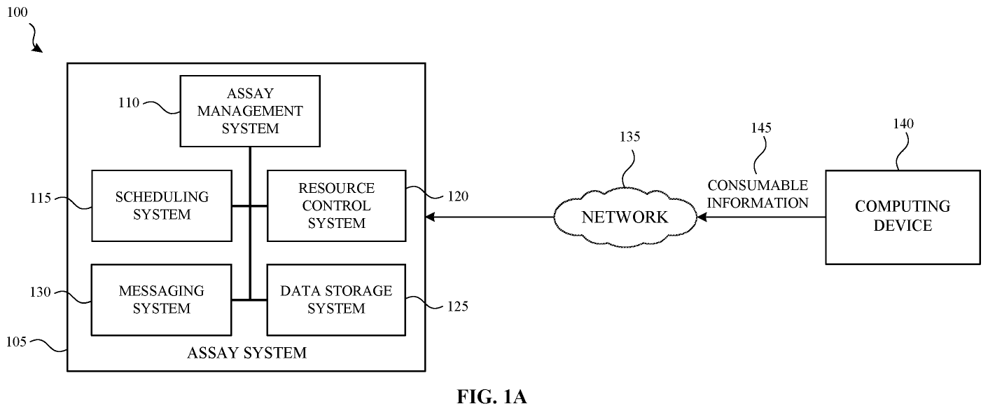

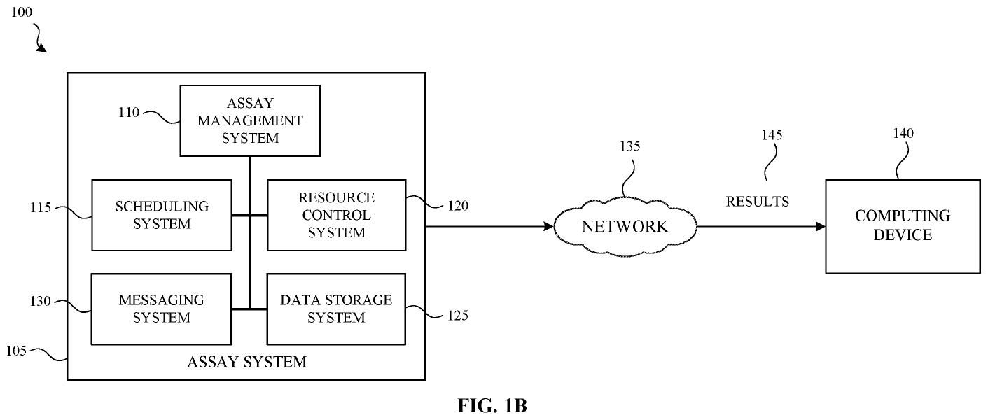

These examples and more will be described in more detail with respect to FIG. 1A-FIG. 4. Additionally, the following terms may be understood by those of skill in the art. However, for purposes of clarification the following terms should be given the following meaning as used herein. As used herein, an “assay” is a test comprising one or more procedural steps applied to a sample in order to make determinations concerning one or more analytical targets. Examples of such determinations include, but are not limited to, whether or not the analytical target is present in the sample, the concentration of the analytical target in the sample, biochemical or immunological activity exhibited by the target, information about the chemical composition of the target, or other physical, chemical, or biochemical properties of the target. Example procedural steps may include, separately or in combination, introducing a reagent to the sample, causing a chemical reaction to take place in the sample, applying electromagnetic radiation to the sample, taking an image of the sample, heating the sample, measuring a physical property of the sample, or performing any other physical or chemical activity necessary to obtain any result necessary to make the determinations. Example analytical targets include proteins, nucleic acids, biological cells, microorganisms, chemical molecules, and chemical elements. For example, the assay may be coupled with one or more reagents which, in tandem or separately, measures the biochemical or immunological activity of one specific biochemical or immunological target within the sample.

As used herein, a “mode” or a “modality” consists of a set of procedural steps. In some examples, the set of procedural steps share a common set of hardware and/or algorithm requirements. As used herein, “multimodal” means more than one mode. Multimodal may apply to two or more different assays, two or more similar assays and/or two or more of the same assays.

As used herein, the term “multiplex” or “multiplex system” is a system that simultaneously or substantially simultaneously uses resource items to carry out assay process steps, and/or communicates (e.g., via a communication channel) the results of the processes steps such that at least two of those process steps take place in a time period that overlap or at least partially overlap.

A communication channel may be a communication channel between at least one computing device and at least one assay analysis systems, a communication channel between various subsystems and/or resource items, or other means by which information may be transferred from one system to another including, but not limited to, a fluorescent signal, Bluetooth, infrared, and the like. Additionally, a channel may include any type of channel including, but not limited to, a communication channel (such as described above) and/or any type of sensing that is performed by the assay analysis system. For example, a multiplex fluorescent assay can send multiple pieces of information across a “visual channel” even if a particular signal is in a different wavelength and/or is being measured by a separate (e.g., visual) sensor. As used herein, the term “multiplex” or “multiplex assay” is a procedure coupled with one or more reagents which, in tandem or separately, measure a biochemical or immunological activity of more than one biochemical or immunological target within a sample.

Although the examples of the multimode assay system described herein relate to diagnostic testing in a medical setting, it is contemplated that the system and methods described herein may be used across a variety of settings and industries. Additionally, although the examples described herein reference two assays being performed in parallel, the multimode assay system described herein may perform more than two assays in parallel or substantially in parallel.

FIG. 1A illustrates a multimode assay system 100 that receives information from, or is otherwise associated with, a multimode consumable in order to perform multiple assays in parallel according to an example. In an example, the multimode assay system 100 includes at least one assay system 105. The assay system 105 may include various hardware resources or resource items that are used to perform the two or more assays in parallel, substantially in parallel and/or in series. Example resource items include, but are not limited to, one or more pumps, one or more valves, one or more temperature elements, one or more light sources, and one or more sensors (e.g., temperature sensors, image sensors). Although specific resource items are mentioned, the assay system 105 may include additional resource items. For example, the assay system 105 may include a slot or port for receiving a multimode consumable.

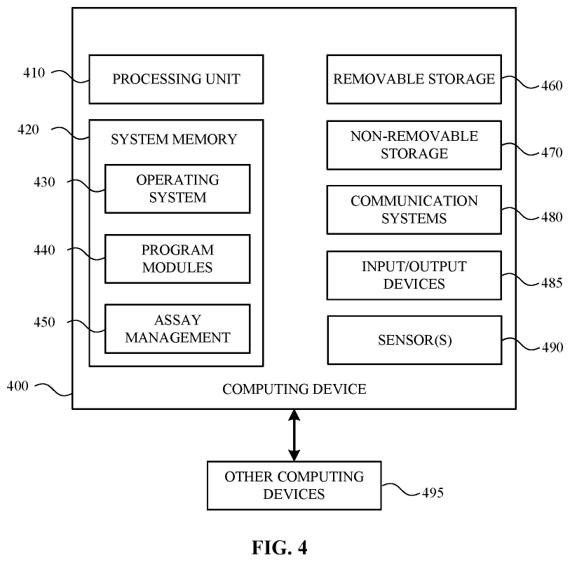

In an example, the assay system 105 includes at least one assay management system 110, at least one scheduling system 115, at least one resource control system 120, at least one data storage system 125 and at least one messaging system 130. The assay system 105 may also include other components or systems such as, for example, the various systems and components shown and described with respect to FIG. 4.

The multimode assay system 100 also includes or is otherwise associated with at least one computing device 140. The computing device 140 may be a desktop computing device, a portable computing device, a mobile phone, a tablet computing device and the like. The computing device 140 may be communicatively coupled to the assay system 105. For example, the computing device 140 may be communicatively coupled to the assay system 105 via a network 135. Although a network 135 is specifically shown and described, the computing device 140 and the assay system 105 may be communicatively coupled via any wired and/or wireless communication protocol. In another example, the computing device 140 (or various components of the computing device 140) may be integrated with the multimode assay system 100. Thus, in some examples, the multimode assay system 100 may include the hardware resources required to perform the various assays indicated by a received multimode consumable (or other received instruction) or the multimode assay system 100 may include some or all of the various components and/or systems associated with the computing device 140.

In an example, the computing device 140 may be used to determine and/or provide consumable information 145 to the assay system 105. The consumable information 145 includes information about which assays the assay system 105 will perform.

For example, an operator of the computing device 140 may select a particular multimode consumable and provide information about the multimode consumable to the computing device 140. In one example, the operator of the computing device 140 may manually input the information about the multimode consumable into the computing device 140. In another example, an image sensor, a scanner or other input device (e.g., associated with the computing device 140) may automatically retrieve and/or determine information about the multimode consumable.

In yet another example, the consumable information 145 may be determined by the assay system 105. For example, the multimode consumable may include or otherwise be associated with code (e.g., a bar code, a QR code). The code provides information regarding the multiple assays that will be performed by the assay system 105. Thus, instead of requiring an operator to manually enter information corresponding to which assays are to be performed, the operator may insert the multimode consumable into the slot or port associated with the assay system 105. Once the multimode consumable has been inserted into the slot or port of the assay system 105, an image sensor, a code reader or other such device associated with the assay system 105 may scan or read the code. In another example, the multimode consumable may include at least one processing device that provides instructions to the assay system 105 and/or the computing device 140 regarding which assays are to be performed.

Using this information, the assay system 105 automatically determines or otherwise identifies which assays are to be performed. As the code identifies multiple assays (e.g., either the same assays, similar assays or different assays) that are to be performed, the assay system 105 may begin to schedule the various processes and resource items that will be used to perform each of the assays.

In some examples, two or more predefined assays may be on or otherwise associated with a particular multimode consumable. For example, a first multimode consumable may be manufactured to contain a first plurality of assays (e.g., a polymerase chain reaction (PCR) test and a lateral flow assay (LFA)) while a second multimode consumable may be manufactured to contain a second plurality of assays (e.g., PCR and cytometry). Although specific examples are given, the multimode consumable may include any number of assays in various combinations.

In another example, the multimode consumable may include a data storage system that includes identification information regarding the assays that are to be performed on at least one sample provided on the multimode consumable. In an example, the multimode consumable may be multimode test card such as described in U.S. Patent Publication No. 2022/0203363, the entire disclosure of which is hereby incorporated by reference in its entirety.

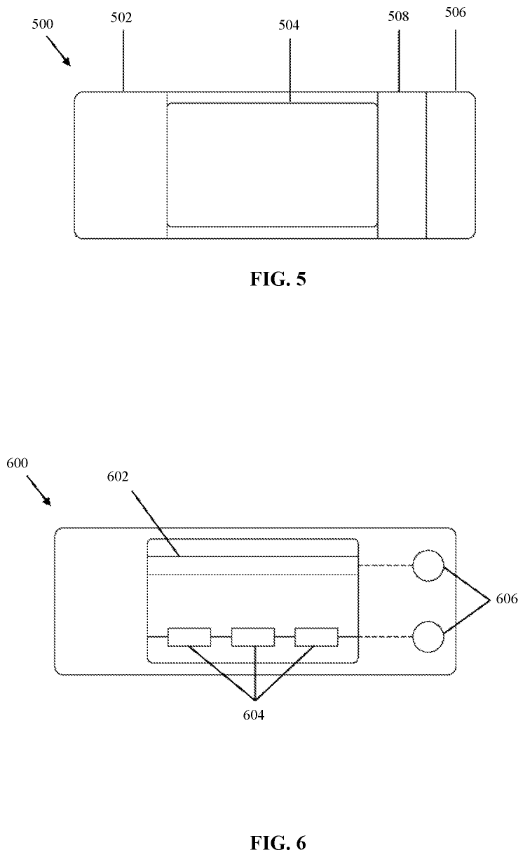

Several example multimode consumables are depicted in FIG. 5-FIG. 9.

A generalized multimodal test card 500 is illustrated in FIG. 5. The test card 500 may be divided into standardized zones to provide a framework for multimode test cards. The test card 500 may include standardized regions that are pre-allocated for a testing device interface 502, an optical measurement zone 504, a sample processing/rehydration zone 508, and a loading port zone 506. The optical measurement zone 504 may be specifically sized to match the viewable area of the optical system of the device used for analysis. The testing device interface zone 502 may contain features for connecting to device electronics, pneumatic and fluid actuating systems, and mechanical features for locating the test card within the analysis device. Mechanical compatibility of the test card with the device may extend beyond this region. For example, portions of the test card that are inserted into the device must be shaped so as to fit within the receptacle of the device. In some embodiments, all portions of the test card except the loading port zone 506 are configured to be insertable into the device used for analysis.

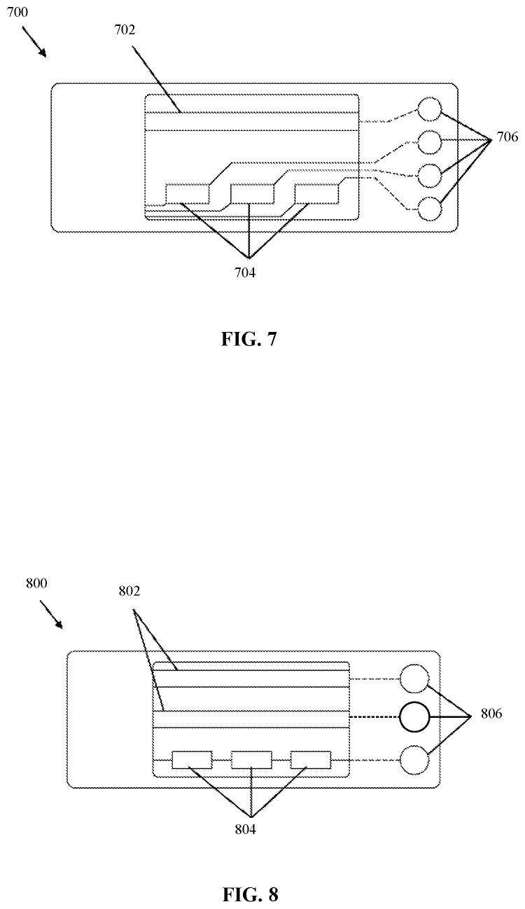

An example multimodal test card 600 configured for PCR and LFA is shown in FIG. 6. Although FIG. 6 (and FIG. 7-FIG. 9) give examples with respect to PCR and LFA, these are for example purposes only. As such, it is contemplated that the multimodal test cards shown and described with respect to FIG. 6-FIG. 9 may be used with other modalities/combinations including, but not limited to cytometry, electrochemistry, along with others listed in Table 1 below. It includes a channel layer with multiple PCR reaction wells 604 and a cavity that accommodates a LFA strip 602. It is configured to perform both PCR and LFA tests. It includes two loading ports 606 to feed both the LFA strip 602 and the PCR wells 604. The PCR sample is loaded into a series of PCR wells (e.g., 3) 604 with the same target.

Another example multimodal test card 700 configured for PCR and LFA is shown in FIG. 7. It includes a channel layer with multiple PCR reaction wells 704 and a cavity that accommodates a LFA strip 702. It is configured to perform both PCR and LFA tests. Each sample is loaded into a different loading port 706. For the PCR part of the test card, each PCR reaction well 704 is fed by its own loading port 706. In some embodiments, the LFA strip 702 contains a single target or multiple targets. This exemplary test card is configured to detect three PCR targets and multiple LFA targets

Another example multimodal test card 800 configured for PCR and LFA is shown in FIG. 8. It includes a channel layer with multiple PCR reaction wells 804 in series and two separate cavities that each accommodate a LFA strip 802 in parallel. This exemplary multimodal test card 800 is configured to perform both PCR and LFA tests. It includes separate loading ports 806 for each LFA strip 802 and the PCR wells 804. The PCR sample is loaded into a series of PCR wells (e.g. 3) 804 with the same target. The test card includes three tests and/or modes. In some embodiments, such a test card is useful for COVID-19 testing. In some embodiments, the PCR tests provide virus genetic information, the first LFA provides virus antigen information, and the second LFA provides antibody information.

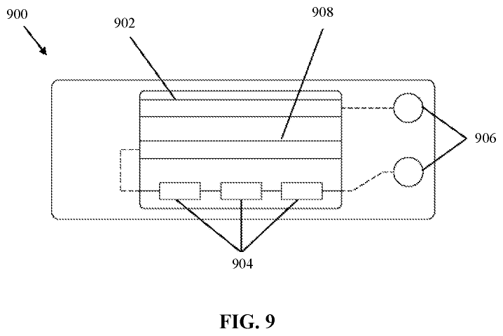

Another example multimodal test card 900 configured for PCR and LFA is shown in FIG. 9. It includes a channel layer with multiple PCR reaction wells 904 in series and two separate cavities that each accommodate a LFA strip 902 and 908. It is configured to perform both PCR and LFA tests. It includes separate loading ports 906 for one of the LFA strips 902 and for the PCR tests. The first LFA strip 902 is an independent LFA strip with its own loading port. The second LFA strip 908 is a PCR dependent LFA strip and is connected in series with the PCR wells 904. The sample loaded in the PCR loading port 906 is pulled in the PCR wells 904 then in the LFA strip 908. Table 1 below gives some other examples of possible tests (various combinations of the tests listed below are contemplated).

TABLE 1
Sample Tests for Multimodal Test Cards

| Test 1 | Test 2 | Test 3 |
|---|---|---|
| Nucleic acid | Nucleic acid | None |
| amplification | amplification | |
| Lateral flow assay | Lateral flow assay | Nucleic acid |
| | | amplification |
| Assay | Assay | Lateral flow assay |
| Antibody | Antibody | Assay |
| Colorimetric | Colorimetric | Antibody |
| Turbidity | Turbidity | Colorimetric |
| Viscosity | Viscosity | Turbidity |
| Light scattering | Light scattering | Viscosity |
| Cytometry | Cytometry | Light scattering |
| Chemistry | Chemistry | Cytometry |
| — | — | Chemistry |

Referring back to FIG. 1 and as indicated above, each multimode consumable is associated with at least one assay or a plurality of assays. As such, when the multimode consumable is selected and prepared with an appropriate sample (or samples), information about the multimode consumable (e.g., consumable information 145) is provided to the computing device 140. The computing device 140 may subsequently provide the consumable information 145 to the assay system 105 via the network 135 or other communication method.

When the consumable information 145 is received, the assay management system 110 uses the consumable information 145 to determine which assays will be performed by the assay system 105. For example, when the consumable information 145 is received, the assay management system 110 may access the data storage system 125 and use the consumable information 145 to determine or otherwise identify the assays to be performed as well as an “assay recipe” for each assay.

In another example, the computing device 140 receives the consumable information 145 (either from user input or via the assay system 105). In such an example, the computing device 140 determines the assays to be performed and performs various operations based on that determination. For example, the computing device 140 may generate or otherwise obtain schedule information, assay recipes and so on. Once this information is generated or otherwise obtained, the computing device 140 may provide this information to the assay system 105.

The assay recipe may be static or dynamic. For example, the assay recipe may be designed based on a particular assay that is to be performed. As such, the assay recipe may be provided on a multimode consumable or otherwise be provided along with the consumable information 145. In such an example, the schedule associated with the assay recipe may be determined in advance of the assay being performed. In another example, the assay recipe may be determined dynamically. In yet another example, a static assay recipe may be dynamically updated based on, for example, one or more operating conditions of the assay system 105.

In an example, an assay recipe includes a number of operations or steps that are required to be performed in a particular order and for a particular duration of time. Additionally, each operation or step may require the use of one or more resource items. The assay recipe may also include a priority for each operation or step and/or a tolerance (e.g., a variation in an order and/or timing of a particular operation or step) for each operation or step. In an example, some or all of this information is stored in the data storage system 125. In another example, some or all of this information may be included in the consumable information 145.

When the assay recipe for each assay is identified or otherwise determined, the assay management system 110 generates a process flow for each assay based on the assay recipe. For example, the assay recipe for a first assay may require five operations or steps, with each operation or step requiring the use of a particular resource item for a minimum duration of time. Likewise, the assay recipe for a second assay may require four operations or steps, with each operation or step requiring the use of a particular resource item for a minimum duration of time.

The assay management system 110 may use the process flow associated with each assay recipe to track the status of each operation or step. For example, the process flow may be used to track when a particular operation or step starts, when the particular operation or step has been completed, an expected time duration associated with a particular operation or step, a priority of an assay and/or a priority of an operation or step in a particular assay recipe as well as a tolerance associated with each operation or step. Once a particular operation or step for either assay recipe has been completed, the assay management system 110 may cause the next operation or step for that assay recipe to commence. Likewise, when a particular operation or step for the other assay recipe has been completed, the assay management system 110 may cause the next operation or step for that assay recipe to commence.

When the assay management system 110 generates a process flow for the first assay and the second assay, the assay management system 110 communicates with the scheduling system 115 to generate an initial resource schedule. For example, the assay management system 110 may provide a resource item request associated with a first operation or step in the first assay recipe to the scheduling system 115 and/or may provide a resource item request for a first operation or step in the second assay recipe to the scheduling system 115.

In an example, the resource item request may include a resource identifier associated with the requested resource item and timing information associated with the requested resource item. In an example, the timing information may include a use period of the resource item, a tolerance associated with the use period (e.g., a start time, an end time, a resource item use duration) of the resource item and/or a priority associated with the use period of the resource item.

Once this information is received, the scheduling system 115 determines whether the requested resource item is available for the requested time. In making this determination, the scheduling system 115 may access a resource schedule that is stored in the data storage system 125. In an example, the resource schedule includes information about when each resource item associated with the assay system 105 is available and/or unavailable. In this example, the resource schedule is initially empty as performance of each assay has not yet commenced.

Continuing with the example above, the assay management system 110 provides a first resource item request to the scheduling system. In this example, the first resource item request is associated with a first operation of the first assay recipe. As indicated above, the first resource item request may include: 1) a resource identifier associated with the requested resource item; and 2) timing information associated with the requested resource item.

Once this information is received, the scheduling system 115 accesses the resource schedule and determines whether the requested resource item is available for the requested time. As the resource schedule is initially empty, all resource items are available. As such, the scheduling system 115 acknowledges the first resource item request and updates the resource schedule. In this example, the resource schedule now includes the resource identifier and/or the timing information associated with the first operation of the first assay recipe.

In an example, the assay management system 110 determines or otherwise identifies an estimated amount of time that is required to complete each assay and provides the first resource item request to the scheduling system 115 based on the estimated amount of time. For example, if the assay management system 110 determines that the first assay will take twenty minutes to complete and the second assay will take fifteen minutes to complete, the assay management system 110 will submit resource item requests associated with the first assay before submitting resource item request associated with the second assay. In another example, the assay management system 110 may consider the amount of operations or steps associated with each assay recipe when determining which resource item request to submit first. In yet another example, the assay management system 110 may consider a priority of an assay when determining which resource item request to submit first.

Once the initial resource schedule has been generated, the assay system 105 may begin performing the assay. As such, the scheduling system 115 provides instructions to the resource control system 120 to begin utilizing the identified resource item to perform the operation specified by the first assay recipe.

This process may continue, in parallel, for each step or operation associated with the first assay and the second assay. For example, once the initial resource schedule has been generated, the assay management system 110 may submit a second resource item request to the scheduling system 115. The second resource item request may be associated with the first assay recipe or it may be associated with the second assay recipe.

In an example, the assay management system 110 tracks, in real time or substantially real time, the status of each operation or step of each assay recipe to determine when to submit a subsequent operation or step of each assay recipe to the scheduling system 115. For example, when the first operation or step in the first assay recipe is completed (or will be completed within a threshold amount of time) by the assay system 105, the assay management system 110 provides the second operation or step in the first assay recipe to the scheduling system 115. Likewise, when the first operation or step in the second assay recipe is completed (or will be completed within a threshold amount of time) by the assay system 105, the assay management system 110 provides the second operation or step in the second assay recipe to the scheduling system 115.

As each operation or step is received by the scheduling system 115, the scheduling system 115 accesses the resource schedule, determines whether the resource item is available and communicates the availability of the hardware resource back to the assay management system 110. Additionally, the scheduling system 115 instructs the resource control system 120 to utilize the various resource items at the requested times and/or for the requested duration(s) of time as outlined in the resource schedule.

In some examples, the assay management system 110 may communicate with the scheduling system 115 when each operation or step of each assay recipe has been performed (or will be performed within a threshold amount of time). Thus, as one operation or step of the first assay recipe is completed, the assay management system 110 provides a subsequent operation or step of the first assay recipe to the scheduling system 115. In such an example, the scheduling system 115 may access and/or update the resource schedule upon receipt of each new resource item request.

In another example, the assay management system 110 may provide the entire first assay recipe (or portions of the first assay recipe) and/or the entire second assay recipe (or portions of the second assay recipe) to the scheduling system 115 before any operations or steps are completed. In such an example, the scheduling system 115 may generate an initial, complete resource schedule. However, as each operation or step for each assay recipe is completed, the scheduling system 115 and the assay management system may need to determine whether the resource schedule is valid and/or needs to be updated based on, for example, whether performance of the assays remains on schedule (e.g., whether some or all of the operations for each assay recipe were performed or are being performed in the time period initially specified).

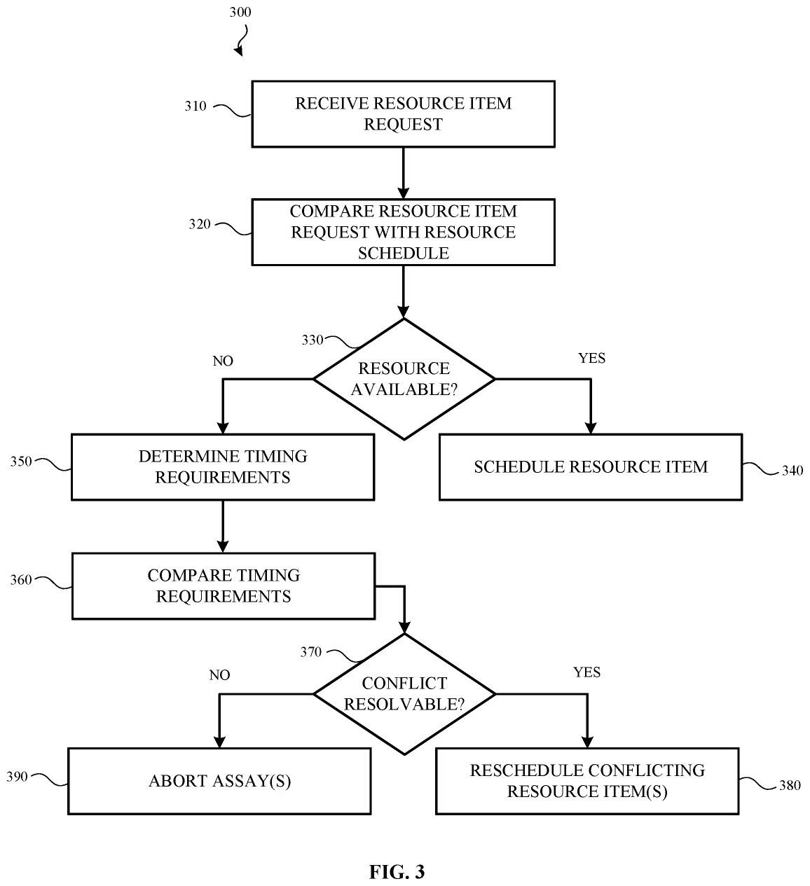

In a multimode assay system 100, such as the one described, it is anticipated that one or more resource item conflicts may arise. For example, the first assay may require a particular resource item (e.g., an image sensor) at a first time period and for a first duration of time and the second assay may require use of the particular resource item at a second time period for a second duration of time. In this example, the first duration of time and/or the first time period may overlap or otherwise conflict with the second duration of time and/or the second time period. In order to address the above, the assay management system 110, in conjunction with the scheduling system 115, is configured to determine whether resource item conflicts exist, determine whether the conflicts are resolvable, and to ultimately resolve the resource item conflict. This process will be described in more detail with respect to FIG. 3.

When each assay has been performed, the messaging system 130 may provide the results 145 of each assay to the computing device 140 via the network 135 such as shown in FIG. 1B.

The following example illustrates an example of a multimodal test card (e.g., multimodal test card 600 (FIG. 6)) may be configured for PCR and LFA.

In this example, an individual obtains a test card 600 and uses the two loading ports 606 to feed the LFA strip 602 and the PCR wells 604. The multimode test card 600 is presented to the assay system 105, for example through a suitable slot or port for receiving it and the consumable information 145 is provided to the computing device 140. In this example, the consumable information 145 specifies that the multimodal test card 600 is configured for PCR and LFA. The consumable information 145 may also specify the assay targets.

Using the consumable information 145, the assay management system 110 determines that the requested assays are PCR and LFA tests. The assay management system 110 also determines an “assay recipe” for each assay. For example, simple assay recipes for PCR and LFA may comprise the following operations and resource items outlined in Table 2 below.

TABLE 2
Assay Modality Resource Utilization Table

| Assay | Step | Resource Items |
|---|---|---|
| PCR and LFA step 0 | Card lock | Limit Switch, Motor |
| PCR step 1 | Fluid loading | Valve, Pump |
| PCR step 2 | Imaging | Light Source, Image Sensor |
| PCR step 3 | Heating | Temperature Element |
| PCR step 4 | Imaging | Light Source, Image Sensor |
| PCR step 5 | Analysis | CPU |
| LFA step 1 | Imaging | Light Source, Image Sensor |
| LFA step 2 | Delay | CPU |
| LFA step 3 | Imaging | Light Source, Image Sensor |
| LFA step 4 | Analysis | CPU |
| PCR and LFA final step | Card release | Limit Switch, Motor |

The assay management system 110 may generate generates a process flow for each assay specified in the consumable information 145. The assay management system may also communicate with the scheduling system 115 to generate an initial resource schedule.

For example, when a single PCR and a single LFA test are performed, each with the same assay target, the resource schedule may constitute dividing a time period covered by each process into a series of smaller time periods. Within each time period, each resource item is given instructions regarding which steps of the different assays it should be performing. This is further shown in Table 3 below:

TABLE 3
Multimode Parallel Resource Schedule Example

| Time | Limit | | | | Light | Image | Temperature | |
|---|---|---|---|---|---|---|---|---|
| Period | Switch | Motor | Valve | Pump | Source | Sensor | Element | CPU |
| 0 to | PCR/LFA | PCR/LFA | — | — | — | — | — | — |
| t1 | Step 0 | Step 0 | | | | | | |
| t1 to | — | — | PCR | PCR | LFA | LFA | — | — |
| t2 | | | Step 1 | Step 1 | Step 1 | Step 1 | | |
| t2 to | — | — | — | — | PCR | PCR | — | LFA |
| t3 | | | | | Step 2 | Step 2 | | Step 2 |
| t3 to | — | — | — | — | — | — | PCR | — |
| t4 | | | | | | | Step 3 | |
| t4 to | — | — | — | — | PCR | PCR | — | — |
| t5 | | | | | Step 4 | Step 4 | | |
| t5 to | — | — | — | — | LFA | LFA | — | — |
| t6 | | | | | Step 3 | Step 3 | | |
| t6 to | — | — | — | — | — | — | — | PCR |
| t7 | | | | | | | | step 5 |
| t7 to | — | — | — | — | — | — | — | LFA |
| t8 | | | | | | | | step 4 |
| t8 to | PCR/LFA | PCR/LFA | — | — | — | — | — | — |
| t9 | Final | Final | | | | | | |
| | Step | Step | | | | | | |

As shown in Table 3, the multimode LFA/PCR tests take approximately 9 time periods. It should be noted that during times t4-t6, PCR was prioritized over LFA due to a resource conflict. The same is true in time t6-t8.

Table 4 below illustrates an example of a multimode serial schedule for PCR and LFA tests. As shown in Table 4, the serial nature requires additional time periods (e.g., eleven time periods) when compared with the multimode parallel resource schedule example shown in Table 3.

TABLE 4
Multimode Serial Resource Schedule Example

| Time | Limit | | | | Light | Image | Temperature | |
|---|---|---|---|---|---|---|---|---|
| Period | Switch | Motor | Valve | Pump | Source | Sensor | Element | CPU |
| 0 to t1 | PCR/LFA | PCR/LFA | — | — | — | — | — | — |
| | Step 0 | Step 0 | | | | | | |
| t1 to t2 | — | — | PCR | PCR | — | — | — | — |
| | | | Step 1 | Step 1 | | | | |
| t2 to t3 | — | — | — | — | PCR | PCR | — | — |
| | | | | | Step 2 | Step 2 | | |
| t3 to t4 | — | — | — | — | — | — | PCR | — |
| | | | | | | | Step 3 | |
| t4 to t5 | — | — | — | — | PCR | PCR | — | — |
| | | | | | Step 4 | Step 4 | | |
| t5 to t6 | — | — | — | — | — | — | — | PCR |
| | | | | | | | | Step 5 |
| t6 to t7 | — | — | — | — | LFA | LFA | — | — |
| | | | | | Step 1 | Step 1 | | |
| t7 to t8 | — | — | — | — | — | — | — | LFA |
| | | | | | | | | Step 2 |
| t8 to t9 | — | — | — | — | LFA | LFA | — | — |
| | | | | | Step 3 | Step 3 | | |
| t9 to t10 | — | — | — | — | — | — | — | LFA |
| | | | | | | | | Step 4 |
| t10 to t11 | PCR/LFA | PCR/LFA | — | — | — | — | — | — |
| | Final Step | Final Step | | | | | | |

Table 5 illustrates a non-multimode PCR schedule according to an example. As shown in Table 5, conducting a PCR test alone, requires eight time periods.

TABLE 5
Non-Multimode PCR Schedule Example

| Time | Limit | | | | Light | Image | Temperature | |
|---|---|---|---|---|---|---|---|---|
| Period | Switch | Motor | Valve | Pump | Source | Sensor | Element | CPU |
| 0 to t1 | PCR | PCR | — | — | — | — | — | — |
| | Step 0 | Step 0 | | | | | | |
| t1 to t2 | — | — | PCR | PCR | — | — | — | — |
| | | | Step 1 | Step 1 | | | | |
| t2 to t3 | — | — | — | — | PCR | PCR | — | — |
| | | | | | Step 2 | Step 2 | | |
| t3 to t4 | — | — | — | — | — | — | PCR | — |
| | | | | | | | Step 3 | |
| t4 to t5 | — | — | — | — | PCR | PCR | — | — |
| | | | | | Step 4 | Step 4 | | |
| t5 to t6 | — | — | — | — | — | — | — | PCR |
| | | | | | | | | Step 5 |
| t7 to t8 | PCR | PCR | — | — | — | — | — | — |
| | Final | Final | | | | | | |
| | Step | Step | | | | | | |

Table 6 illustrates a non-multimode LFA schedule according to an example. As shown in Table 6, conducting a LFA test alone, requires seven time periods.

TABLE 6
Non-Multimode LFA Schedule Example

| Time | Limit | | | | Light | Image | Temperature | |
|---|---|---|---|---|---|---|---|---|
| Period | Switch | Motor | Valve | Pump | Source | Sensor | Element | CPU |
| 0 to t1 | LFA | LFA | — | — | — | — | — | — |
| | Step 0 | Step 0 | | | | | | |
| t2 to t3 | — | — | — | — | LFA | LFA | — | — |
| | | | | | Step 1 | Step 1 | | |
| t3 to t4 | — | — | — | — | — | — | — | LFA |
| | | | | | | | | Step 2 |
| t4 to t5 | — | — | — | — | LFA | LFA | — | — |
| | | | | | Step 3 | Step 3 | | |
| t5 to t6 | — | — | — | — | — | — | — | LFA |
| | | | | | | | | Step 4 |
| t6 to t7 | LFA | LFA | — | — | — | — | — | — |
| | Final | Final | | | | | | |
| | Step | Step | | | | | | |

The examples above are merely a human-readable example of a resource schedule and do not necessarily reflect the formatting of the actual resource schedule generated. However, based on comparing the various tables, it is easy to see how the scheduler works, how conflicts are resolved and how a multimode parallel resource schedule is more efficient than running two assays in series (eleven time periods) and/or separately (a combined fifteen time periods). Additionally, although PCR and LFA are used in the tables above, these are for illustrative purposes only and other assays/tests may be combined.

The assay system 105 may then carry out the recipe as outlined in the resource schedule and utilize each resource item at the time specified in to carry out the assay step specified.

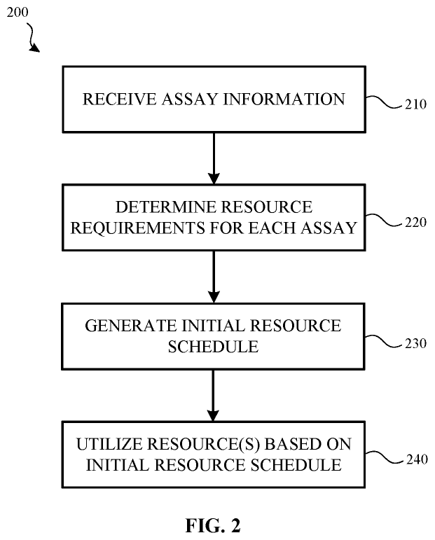

FIG. 2 illustrates a method 200 for generating an initial resource schedule for a multimode assay system according to an example. The method 200 may be performed by an assay system, or one or more subsystems of an assay system, such as, for example, assay system 105 and/or a computing device, such as, for example, computing device 140 shown and described with respect to FIG. 1A.

Method 200 begins when assay information is received (210) by the assay system. In an example, the assay information is used to identify a plurality of assays that will be performed, simultaneously or substantially simultaneously, by the assay system. In an example, the assay information may be associated with and/or provided by a multimode consumable (e.g., a test card).

Once the assay information is received, the assay system determines (220) resource requirements for each assay. For example, the assay information may be used by the assay system to determine or otherwise identify an assay recipe for each assay. In an example, the assay recipe includes a number of operations or steps that are required to be performed by various resource items of the assay system. The operations or steps may need to be completed or performed in a particular order and for a particular duration of time. As explained above, the assay recipe may also include a priority for each operation or step and/or a tolerance for each operation or step.

When the resource requirements for each assay are determined, the assay system generates (230) an initial resource schedule. As explained above, the initial resource schedule may be generated based, at least in part, on a resource item request associated with a first operation of a first assay recipe associated with a first assay of the plurality of assays and/or on a resource item request associated with a first operation or step of a second assay recipe associated with a second assay of the plurality of assays.

The resource item request may include a resource identifier and/or timing information associated with the requested resource item. In this example, the timing information may include a use period of the resource item, a tolerance associated with the use period of the resource item and/or a priority associated with the use period of the resource item.

The assay system uses the resource item request to determine whether the requested resource item is available for the requested time. For example, the assay system may access a resource schedule to determine whether the requested resource item is available. As discussed above, the resource schedule is initially empty as performance of any of the assays has yet to commence.

When the initial resource schedule has been generated, the assay system may utilize (240) one or more of the resource items of the assay system to perform a required operation or step of the assay. In an example, the one or more resource items are utilized based, at least in part, on the timing information associated with the requested resource item.

In some examples, a resource item request may not be able to be fulfilled. For example, when generating the initial resource schedule, it may be determined that a particular resource item is unavailable. In such an example, the scheduling system may determine that the requested resource item is unavailable and determine whether one or more operations may be moved or reordered (e.g., based on timing information associated with the particular resource item and/or an assay recipe). Thus, the initial resource schedule may be updated and/or restarted before completion (e.g., during generation of the initial resource schedule) or upon completion (e.g., once the initial resource schedule is complete the scheduling system may verify each resource item is appropriately scheduled and available based on the assay recipe).

FIG. 3 illustrates a method 300 for updating a resource schedule associated with a multimode assay system according to an example. The method 300 may be performed by an assay system such as, for example, the assay system 105 shown and described with respect to FIG. 1A-FIG. 1B. In an example, the method 300 may be a continuation of the method 200 shown and described with respect to FIG. 2. For example, the method 300 may be performed once an initial resource schedule has been generated. In another example, some or all of the operations shown and described with respect to method 300 may be performed at any time during performance of multiple assays.

Method 300 begins when a resource item request is received (310). In an example, the resource item request may be associated with a first assay recipe or a second assay recipe. The resource item request may include a resource identifier and/or timing information associated with the requested resource item. The timing information may include a use period of the resource item, a tolerance associated with the use period of the resource item and/or a priority associated with the use period of the resource item.

When the resource item request is received, the assay system compares (320) the information in the resource item request (e.g., the resource identifier and/or the timing information) with a resource schedule (e.g., the initial resource schedule that was generated by method 200 (FIG. 2)).

For example, an assay management system associated with the assay system may provide the resource item request to a scheduling system associated with the assay system. The scheduling system may compare the resource identifier and the timing information contained in the resource item request with the resource schedule. In an example, the resource schedule is specific to the resource item specified by the resource identifier. The resource schedule includes information regarding when the resource item is being used (or its anticipated, scheduled or upcoming use) and the timing information (e.g., a use period of the resource item, a tolerance associated with the use period and a priority associated with the use period of the resource item) of the resource item.

As indicated above, the scheduling system may include or otherwise have access to separate resource schedules for each resource item. Thus, when a resource item request is received, the scheduling system may identify the requested resource item (e.g., using the resource identifier), access the resource schedule associated with the resource item and determine whether the resource item is currently being used or is scheduled to be used during the time frame specified in the resource item request. In another example, the scheduling system may maintain a single resource schedule for all of the resource items of the assay system.

Based on the comparison, a determination (330) is made as to whether the requested resource item is available. If the scheduling system determines, based on the resource schedule, that the resource item is available for the requested time and duration, the scheduling system schedules (340) the resource item for the requested time and duration. As part of this process, the scheduling system may update the resource schedule and/or provide confirmation to the assay management system. The method 300 may then repeat when a subsequent resource item request is received.

However, if the scheduling system determines (330) that the resource item is not available (e.g., the requested resource item is currently in use or is scheduled to be in use based on information associated with a first assay recipe), the scheduling system determines (350) the timing requirements for the particular resource item. For example, the scheduling system determines the timing requirements of the resource item in the resource item request and the timing requirements of the resource item in the resource schedule. That is, the scheduling system compares the use period, the tolerance and/or a priority associated with the use period of the resource item associated with the resource item request (e.g., the resource item request received in operation 310) to the use period, the tolerance and/or the priority associated with the use period of the resource item in the resource schedule.

Once this information is determined, the scheduling system and/or the assay management system compares (360) the timing requirements to determine whether the conflict is resolvable. For example, the scheduling system and/or the assay management system may determine, based on information contained in the resource schedule, that the resource item is currently scheduled (e.g., based on a resource item request associated with a first assay recipe) for use at a first time period and for a first duration of time. However, it may also be determined that the time period of the resource item has a tolerance of +/−twenty seconds. For example, the resource item may begin performing a particular operation or step specified in the first assay recipe twenty seconds earlier than originally scheduled or the resource item may begin performance of the particular operation or step specified in the first assay recipe twenty seconds later than originally scheduled.

The scheduling system and/or the assay management system may also determine that the resource item was scheduled to be in use for ten seconds. However, the usage time of the resource item may also be associated with a tolerance. For example, although the usage time of the resource item was scheduled for ten seconds, the resource item may complete the operation in five seconds.

Likewise, the scheduling system and/or the assay management system may also determine, based on information contained in the resource item request (e.g., a resource item request associated with a second assay recipe), that the resource item is requested for use at a second time period and for a second duration of time. However, the second time period and/or the second period of time overlaps (at least partially) with the first time period and/or the first duration of time.

In this example, the time period of the resource item associated with the second assay recipe may have a tolerance of +/−five seconds (e.g., the resource item may begin performing the particular operation or step specified in the second assay recipe five seconds earlier than originally scheduled or the resource item may begin performance of the particular operation or step specified in the second assay recipe five seconds later than originally scheduled). It may also be determined that the priority of this particular operation or step of the second assay recipe is greater/higher than the priority of the particular operation or step specified in the first assay recipe. It may also be determined that the operation or step required by the second assay recipe takes ten seconds to complete but there is not a tolerance associated with this particular operation or step. Thus, this particular operation or step will require at least ten seconds to complete.

Based on the comparison, the scheduling system and/or the assay management system may determine (370) whether the resource item conflict is resolvable. For example, using some or all of the information outlined above, the scheduling system and/or the assay management system may determine that if the resource item is utilized for the performance of the operation or step specified in the second assay recipe five seconds earlier than originally requested and the resource item is utilized for the performance of the operation or step specified in the first assay recipe twenty seconds later than originally scheduled, the assay system can serially perform both operations with the same resource item. Accordingly, the scheduling system and/or the assay management system reschedules (380) the conflicting resource items and/or updates the resource schedule.

For example, the scheduling system and/or the assay management system causes (e.g., by providing instructions to a resource control system) the resource item to perform the operation or step associated with the second assay recipe five seconds earlier than originally requested and also causes the resource item to perform the operation or step associated with the first assay recipe twenty seconds later than originally scheduled. In an example, the resource schedule is dynamically updated to reflect this change. The method 300 may then be repeated until completion of each assay.

In the example above, the entire tolerances for both assay recipes were considered and used for rescheduling. However, in other examples, some or part of the tolerances may be used to determine how to reschedule the conflicting resource items. In another example, because the operation or step associated with the second assay recipe had a higher priority than the operation or step associated with the first assay recipe, the scheduling system may determine to utilize the entire tolerance associated with the first assay recipe without utilizing the tolerance associated with the second assay recipe.

Referring back to operation 370, if it is determined (370) that the resource item conflict is not resolvable, the scheduling system and/or the assay analysis system may abort (390) performance of one or more of the assays. In an example, it may be possible to abort performance of one of the assays while completing performance of one or more of the remaining assays.

For example, if a priority of one or more operations or steps associated with the second assay recipe is greater/higher than a priority of one or more operations or steps associated with the first assay recipe (or an overall priority associated with a particular assay is greater than a priority associated with another assay), the scheduling system and/or the assay management system may abort the assay with the lower priority in order to complete performance of the other assay. In another example, the priority of one assay or operation associated with an assay may be based on received input.

FIG. 4 is a system diagram of a computing device 400 according to an example. The computing device 400, or various components and systems of the computing device 400, may be integrated or associated with an assay system (e.g., assay system 105 (FIG. 1A-FIG. 1B) and/or a computing device (e.g., computing device 140 (FIG. 1A-FIG. 1)). As shown in FIG. 4, the physical components (e.g., hardware) of the computing device are illustrated and these physical components may be used to practice the various aspects of the present disclosure.

The computing device 400 may include at least one processing unit 410 and a system memory 420. The system memory 420 may include, but is not limited to, volatile storage (e.g., random access memory), non-volatile storage (e.g., read-only memory), flash memory, or any combination of such memories. The system memory 420 may also include an operating system 430 that controls the operation of the computing device 400 and one or more program modules 440. In an example, the operating system 430 may be a pre-empting operating system or a non pre-empting operating system. The program modules 440 may be responsible for executing the various assay management 450 methods described herein. A number of different program modules and data files may be stored in the system memory 420. While executing on the processing unit 410, the program modules 440 may perform the various processes described above.

The computing device 400 may also have additional features or functionality. For example, the computing device 400 may include additional data storage devices (e.g., removable and/or non-removable storage devices) such as, for example, magnetic disks, optical disks, or tape. These additional storage devices are labeled as a removable storage 460 and a non-removable storage 470.

Examples of the disclosure may also be practiced in an electrical circuit comprising discrete electronic elements, packaged or integrated electronic chips containing logic gates, a circuit utilizing a microprocessor, or on a single chip containing electronic elements or microprocessors. For example, examples of the disclosure may be practiced via a system-on-a-chip (SOC) where each or many of the components illustrated in FIG. 4 may be integrated onto a single integrated circuit. Such a SOC device may include one or more processing units, graphics units, communications units, system virtualization units and various application functionality all of which are integrated (or “burned”) onto the chip substrate as a single integrated circuit.

When operating via a SOC, the functionality, described herein, may be operated via application-specific logic integrated with other components of the computing device 400 on the single integrated circuit (chip). The disclosure may also be practiced using other technologies capable of performing logical operations such as, for example, AND, OR, and NOT, including but not limited to mechanical, optical, fluidic, and quantum technologies.

The computing device 400 may include one or more communication systems 480 that enable the computing device 400 to communicate with other computing devices 495 such as, for example, routing engines, gateways, signings systems and the like. Examples of communication systems 480 include, but are not limited to, wireless communications, wired communications, cellular communications, radio frequency (RF) transmitter, receiver, and/or transceiver circuitry, a Controller Area Network (CAN) bus, a universal serial bus (USB), parallel, serial ports, etc.

The computing device 400 may also have one or more input devices and/or one or more output devices shown as input/output devices 490. These input/output devices 490 may include a keyboard, a sound or voice input device, haptic devices, a touch, force and/or swipe input device, a display, speakers, etc. The aforementioned devices are examples and others may be used.

The term computer-readable media as used herein may include computer storage media. Computer storage media may include volatile and nonvolatile, removable and non-removable media implemented in any method or technology for storage of information, such as computer readable instructions, data structures, or program modules.

The system memory 420, the removable storage 460, and the non-removable storage 470 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 400. Any such computer storage media may be part of the computing device 400. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively rearranged, included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. In addition, each of the operations described above may be executed in any order. For example, one operation may be performed before another operation. Additionally, one or more of the disclosed operations may be performed simultaneously or substantially simultaneously.

Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

1. A method, comprising:
receiving identification information associated with at least one multimode consumable;
determining, based at least in part, on the identification information, a plurality of assays to be performed;
determining at least one resource requirement for each assay of the plurality of assays; and
generating an initial resource schedule for each assay based, at least in part, on the at least one determined resource requirement, the initial resource schedule enabling each assay of the plurality of assays to be performed substantially simultaneously.

2. The method of claim 1, wherein the initial resource schedule comprises:
at least one resource identifier associated with at least one resource item; and
timing information associated with the at least one resource item.

3. The method of claim 2, wherein the timing information comprises one or more of:
a use period of the at least one resource item;
a tolerance associated with the use period; and
a priority associated with the use period of the at least one resource item.

4. The method of claim 3, wherein the use period comprises one or more of:
a start time;
an end time; and
a resource item use duration.

5. The method of claim 2, wherein the at least one resource item is a hardware resource of an assay system.

6. The method of claim 1, wherein the multimode consumable is a multimode test card.

7. The method of claim 1, further comprising receiving at least one resource item request associated with a first assay of the plurality of assays, the at least one resource item request comprising:
at least one resource identifier associated with at least one resource item; and
timing information associated with the at least one resource item.

8. The method of claim 7, further comprising:
comparing the at least one resource item request with the initial schedule; and
determining, based at least in part on the comparing, an availability of the at least one resource item.

9. The method of claim 8, further comprising updating the initial schedule based, at least in part, on the determination that the at least one resource item is available.

10. The method of claim 8, further comprising comparing timing information in the at least one resource item request with timing information in the initial schedule based, at least in part, on a determination that the at least one resource item is not available.

11. The method of claim 10, wherein comparing timing information in the at least one resource item request with timing information in the initial schedule comprises one or more of:
comparing a tolerance associated with the use period of the at least one resource item in the initial schedule with a tolerance associated with the use period of the at least one resource item in the at least one resource item request; and
comparing a priority associated with the use period of the at least one resource item in the initial request with a priority associated with the use period of the at least one resource item in the at least one resource item request.

12. The method of claim 10, further comprising updating the initial schedule based, at least in part, on the comparing the timing information in the at least one resource item request with the timing information in the initial schedule.

13. A method, comprising:
receiving at least one resource item request associated with a first assay of a plurality of assays, the plurality of assays being performed in parallel by a single assay system;
determining based, at least in part, on a resource schedule associated with the single assay system, an availability of the at least one resource item; and
updating the resource schedule based, at least in part, on the determined availability of the at least one resource item.

14. The method of claim 13, wherein the at least one resource item request comprises:
a resource identifier associated with at least one resource item; and
timing information associated with the at least one resource item.

15. The method of claim 13, wherein determining an availability of the at least one resource item comprises determining timing information of the at least one resource item, the timing information of the at least one resource item being associated with a second assay of the plurality of assays.

16. The method of claim 13, wherein the resource schedule is updated based, at least in part, on priority information associated with the at least one resource item request.

17. The method of claim 13, wherein the resource schedule is updated based, at least in part, on tolerance information associated with the at least one resource item request.

18. A system, comprising:
a processor; and
a memory communicatively coupled to the processor and storing instructions that, when executed by the processor, perform operations, comprising:
receiving a first resource item request associated with a first assay, the first resource item request identifying at least one resource item;
receiving a second resource item request associated with a second assay, the second resource item request identifying the at least one resource item; and
determining, based at least in part, on first timing information associated with the first resource item request and on second timing information associated with the second resource item request, an availability of the at least one resource item.

19. The system of claim 18, further comprising instructions for allocating the at least one resource item to the first assay and the second assay based, at least in part, on the first timing information associated with the first resource item request and the second timing information associated with the second resource item request.

20. The system of claim 18, further comprising altering the first timing information associated with the first resource item request based, at least in part, on a priority associated with the second timing information associated with the second resource item request.
